(19) United States
(12) Patent Application Publication
(10) Pub. No.: US 2009/0020122 A1
(43) Pub. Date: Jan. 22, 2009

(54) Respiratory device for treating obstructive sleep apnea and method for controlling said device

(76) Inventor: Helmut Hoffrichter, Pampow (DE)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 11/778,223

(22) Filed: Jul. 16, 2007

(51) Int. Cl. A61M 16/00

(52) U.S. Cl. 128/204.23

(57) ABSTRACT

A respirator device for use in treating obstructive sleep apnea is described, with a pressure generator for generating an artificial atmosphere having a treatment pressure, with a pressure regulating device for controlling the pressure generator, and with a respiratory tube with a breathing mask for supplying the artificial atmosphere generated by the pressure generator to a patient, which is characterized in that it has a control mechanism for the continuous determination of a pressure loss occurring over an airway resistance in the airways of the patient, and for the essentially proportional adjustment of the pattern of treatment pressure to the pattern of the determined pressure loss.

The use of a respiratory device of this type for the treatment of sleep apnea substantially decreases the strain on the respiratory musculature of the patient, makes the course of treatment more pleasant, and decreases potential secondary effects of the treatment.

### BACKGROUND OF THE INVENTION

The invention relates to a respiratory device for treating patients who suffer from obstructive sleep apnea, comprising a pressure generator for generating an artificial atmosphere at a treatment pressure, a pressure regulating device for controlling said pressure generator, a respiratory tube with a breathing mask for supplying the artificial atmosphere generated by the pressure generator to a patient, and a method for controlling said device.

Respiratory devices for treating obstructive sleep apnea generate an artificial atmosphere, the pressure of which is higher than the natural atmosphere, and which is supplied continuously, via a respiratory tube and a face mask, during sleep to the patient being treated.

During sleep, as muscle tone generally decreases, the soft tissues of the respiratory passages in the area around the pharynx can collapse, causing the sleeping patient to begin choking. This respiratory arrest is known as obstructive sleep apnea. The reason for the collapse of the soft respiratory passages is a drop in pressure in them, caused by the high rate of respiratory airflow. As a result, the respiratory passages in the area around the base of the tongue and the soft palate are no longer able to withstand the difference in pressure to the exterior, normal surrounding atmosphere. They collapse against one another and obstruct the airway. The continued suction force of the lungs further intensifies the obstruction, and the patient is no longer able to take in any air for several tens of seconds. This process can occur several hundred times during sleep every night. The long-term effects are a decrease in quality of life, diseases of the cardiovascular system, and an overall decrease in life expectancy.

When a patient with obstructive sleep apnea breathes from an artificial atmosphere, the pressure of which is greater than the pressure of the natural surrounding atmosphere, at least by the level of the flow-based drop in pressure in the area around the pharynx, then the soft tissue in the area around the pharynx can no longer collapse. The patient is again able to breathe freely and spontaneously, and the number of sleep apneas is reduced to that of a normal person. The artificial atmosphere required for this is generated by a respiratory device, and is supplied to the patient via a respiratory tube and a breathing mask.

Respiratory devices that generate an artificial atmosphere at a continuously constant treatment pressure are known as CPAP systems (CPAP=Continuous Positive Airway Pressure).

The airflow in the respiratory passages of a patient is not free from resistance. Laminar and turbulent resistance components distributed throughout the passages produce an obstructive effect. Resistance components are those that resist flow in the nasal passages, the soft-walled respiratory passages in the pharynx, and the bronchial system. The degree of turbulent resistance is dependent upon the flow rate.

The airway resistances of a patient and the flow resistances of a device are arranged in series. The internal resistance of a device can be influenced via a corresponding device controller. A low level of strain on the respiratory musculature is achieved when the flow resistance of the device is negatively configured, thereby counteracting the effect of the positive airway resistances of the patient. A negative resistance has a reversed pressure/flow behavior. Referred to the internal flow resistance of a CPAP device, said device would then generate a higher pressure when acted upon by an air flow in the direction of the patient, and would generate a lower pressure when the patient exhales air in the direction of the device.

CPAP devices, which generate a higher treatment pressure during inspiration and a lower treatment pressure during expiration, are known as bi-level devices (e.g. a device under the name BIPAP® of the RESPIRONICS firm). To determine the point of transition from inspiration to expiration and vice versa, these devices have a processor, called the breath trigger. Bi-level devices are always used if the patient already has generally elevated airway resistance due to other illnesses, or if treatment using a CPAP device would be successful only at a very high treatment pressure.

The control of the pressure in bi-level systems is comparable to the control of the pressure in respiratory devices for artificial respiration. One drawback consists in the fact that only two pressure levels exist, which are preset and cannot be adjusted, regardless of the respective depth of respiration. The shifts in pressure initiated via the breath trigger create a rectangular respiratory pressure pattern, and therefore no optimal compensation of the resistances of the respiratory passages. Thus the transition from the lower to the higher pressure level effects a brief period of artificial respiration, in that air is forced into the lungs. During the transition from the higher to the lower pressure level, a constriction of the small respiratory passages can occur due to the high expiration flow at the start, making expiration even harder. These effects are uncomfortable for the patient and can be mitigated by rounding off the transition from one pressure level to the other, rather than structuring it in a precisely rectangular pattern.

The comfort of a bi-level device is decisively influenced by the proper functioning of the breath trigger. Precisely at the end of the expiration phase, when the respiratory air flow has nearly died down, additional air fluctuations occur, because the stroke volume rhythmically influences the volume of the compressed lungs and the flow of air exiting them. Therefore a breath trigger cannot precisely identify the end of an expiration, and the patient has the feeling that the change in pressure frequently takes place at the incorrect time.

### BRIEF SUMMARY OF THE INVENTION

It is thus the object of the invention to provide a respiratory device for treating sleep apnea, which, when used, substantially reduces the strain on the respiratory musculature of the patient, makes the course of treatment more pleasant, and decreases potential secondary effects of the treatment. A further aspect of the invention is to provide a method for controlling such a device.

The respiratory device according to the invention comprises a pressure generator that is capable of providing any level of pressure up to a freely selectable threshold limit, and is controlled via a pressure regulator. With the pressure generator, for example a ventilator, the respiratory device generates an artificial atmosphere, which is supplied to the patient through the respiratory tube and the breathing mask.

The control mechanism contained in the respiratory device according to the invention, which in one advantageous further improvement can be a servo processor, detects a pressure loss from the airway resistance and adjusts the treatment pressure course essentially proportionally to the pattern of the detected pressure loss.

In one advantageous further improvement, the control mechanism is equipped to calculate the pressure loss from the airway resistance using the measured variables of the respiratory airflow and airway resistance of the patient. For this purpose, the respiratory device is equipped with a device for determining respiratory airflow. The patient's airway resistance can be determined externally and communicated to the respiratory device in the form of an input, or, this resistance can automatically be permanently or intermittently determined with the help of a measuring device that is integrated into the respiratory device.

As an advantageous further improvement, a possible continuous or intermittent measurement of airway resistance can be effected, for example, with the help of a modulated pressure or airflow signal. In this manner, changes in airway resistance are always immediately accounted for in the inventive control mechanism of the respiratory device. Because air is ordinarily supplied from the respiratory device via a nasal mask, and the flow resistance in the nose is subject to fairly major changes, the continuous measurement of airway resistance is a good way of compensating for the varying degrees of impairment of nasal respiration.

A further advantageous embodiment of the respiratory device of the invention involves combining the inventive properties with the operating mode for devices that function on the basis of the auto-adjust principle (auto-CPAP) for determining an optimal basic pressure, which are known in the prior art and described in the patent literature, for example in EP 0705 615 A1. A respiratory device equipped with this combination according to the invention then automatically determines the most favorable basic pressure, which can also change with the physical constitution of the patient.

Dispensing with the above-described advantageous embodiments, both basic pressure and airway resistance can be determined via external means and permanently entered into the respiratory device of the invention.

The overall effect achieved with the respiratory device of the invention is that of a respiratory force amplifier, which supports inspiration effort with a matched pressure increase and expiration effort with a likewise matched pressure decrease. This effect is comparable to that of a power steering mechanism or a power braking mechanism in an automobile.

Comfortable breathing is achieved in that both the pressure increase during inspiration and the pressure decrease during expiration are nearly proportional to the pressure loss from airway resistance. To accomplish this, with known airway resistance, the pressure loss is calculated as the product of the airway resistance and a portion of the square of the respiratory airflow, since turbulent resistance increases in a linear fashion with the flow rate. The size of this portion determines the degree of respiratory force amplification.

Also advantageous is an adjustment to the different stages of sleep, which are known to vary according to different respiratory parameters, such as breathing rate and tidal volume. In this manner, at low respiratory intensity, a proportional artificial respiration is prevented in that the difference between maximum inspiration pressure and minimum expiration pressure automatically becomes smaller. At high respiratory intensity, however, the difference between maximum inspiration pressure and minimum expiration pressure increases accordingly.

From a predetermined intensity it can also be established with the help of the control mechanism whether the entire pressure loss from respiratory resistance or only a portion of it should be compensated for via the respiratory device. To accomplish this, the output signal from the control mechanism and the predetermined basic pressure signal are added together and supplied to the pressure regulating device as a command variable.

### BRIEF DESCRIPTION OF THE DRAWINGS

Below, the invention will be described in greater detail with reference to an exemplary embodiment. The attached set of drawings shows:

FIG. 1 a functional block diagram of an exemplary embodiment of a respiratory device according to the invention and

FIG. 2 a chronological representation of the signal patterns for respiratory airflow \dot{V}ν, pressure loss Δp, treatment pressure p_{act} and intrapulmonary pressure p_{L} for different proportions of the pressure loss Δp taken into account in determining the treatment pressure p_{act}.

### DETAILED DESCRIPTION OF THE INVENTION

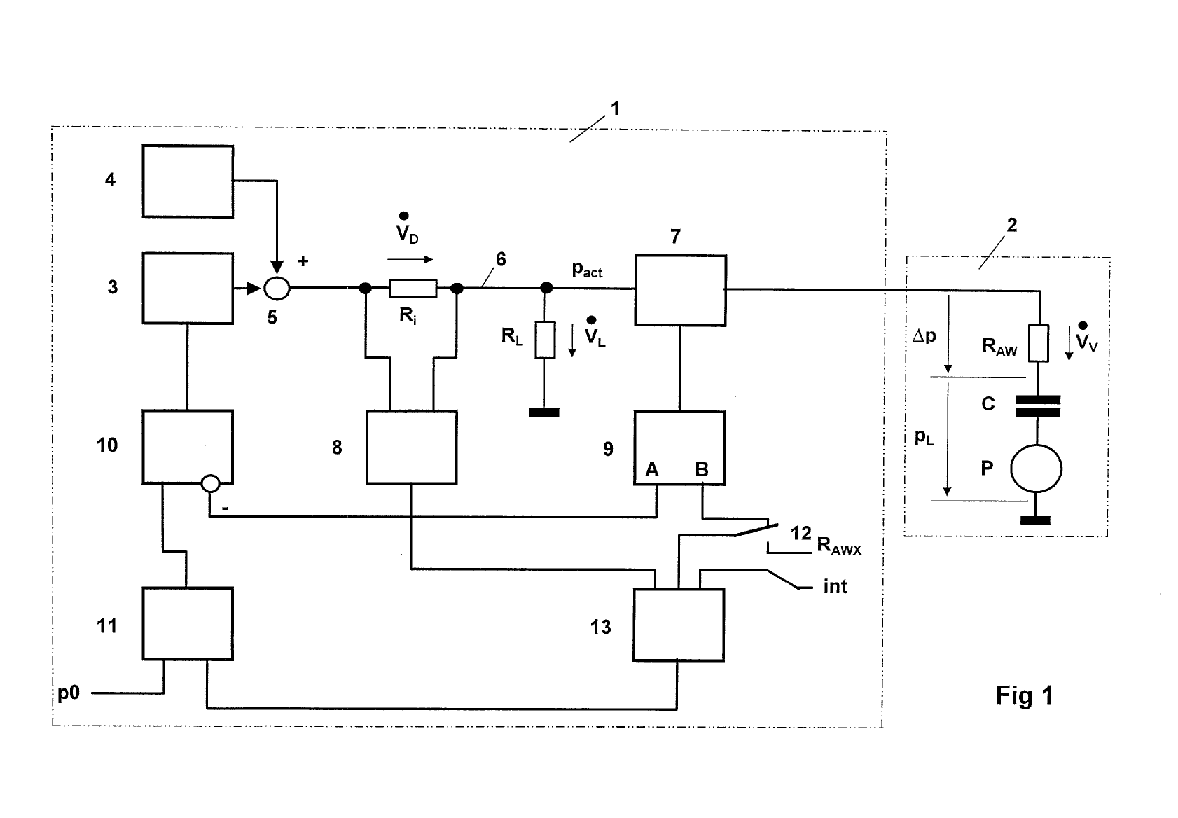

The functional block diagram in FIG. 1 shows a variant that represents one possible implementation of a respiratory device 1 according to the invention.

The respiratory device 1 comprises a pressure generator 3 for generating pressure, here in the form of a ventilator, and an oscillation pump 4, which generates an oscillating pressure pattern with a low amplitude. The two pressure levels are combined in a summation unit 5 to form a total pressure, which is supplied via a flow resistance Rᵢ and a respiratory tube 6 to the breathing mask 7. At the flow resistance Rᵢ, this creates a pressure decrease that is dependent upon the airflow from the device \dot{V}_{D}. A flow processor 8 uses the signal from the device airflow \dot{V}_{D} to determine the signal pattern of the respiratory airflow \dot{V}ν that ventilates the lungs of the patient 2. This is achieved by calculating the pattern of the leakage airflow \dot{V}_{L} and subtracting this from the pattern of the device airflow \dot{V}_{D}. A leakage airflow is a desired airflow through an expiration valve R_{L}, which ordinarily is simply an opening into the surrounding air, and a possible additional parasitic escape of air through portions of the mask that are not airtight.

The treatment pressure p_{act} is measured in the breathing mask 7, which ordinarily is simply a nasal mask. The pressure measured there is supplied to a pressure processor 9, which generates two output signals. One signal at the output A represents the current constant pressure portion and is supplied to the pressure regulating device 10 as the actual pressure. Said device compares the actual pressure with a target pressure, which is supplied by the summation unit 11 and contains as a component the predetermined basic pressure p₀ of the respiratory device 1. The difference between the target pressure and the actual pressure determines the level of pressure to be generated by the pressure generator 3 in such a way that, to the greatest possible extent, no permanent control deviation occurs.

The other signal at the output B of the pressure processor 9 represents the positive-negative pressure in the breathing mask 7. This is generated by the oscillation pump 4. However, its amplitude is dependent upon the size ratios of all existing resistances, the flow resistance Ri, the airway resistance R_{AW} of the patient 2 and the flow resistance R_{L} of an expiration opening. For purposes of clarity the figure shows the compliance C and the respiratory pump P of the patient, however these do not contribute to the functioning of the respiratory device 1 according to the invention.

The variables of flow resistance Rᵢ and flow resistance R_{L} of the expiration opening are known, so that the pressure processor can calculate the differential pressure over the airway resistance R_{AW}, which is a measurement of the magnitude of this resistance.

The measurement result is supplied via the reversing switch 12 to a servo processor 13, which uses the determined respiratory airflow \dot{V}ν to calculate the pressure loss Δp from the airway resistance R_{AW}, and from the predetermined intensity int determines whether the total pressure loss Δp that is consumed via the airway resistance R_{AW}, or only a part of it, should be compensated for via the respiratory device 1.

By actuating a reversing switch 12, the resistance input to the servo processor 13 can be switched over to a manual input of a resistance value R_{AWX}. In this mode of operation, an externally determined airway resistance can be permanently predetermined, which can be practical for certain applications.

The output signal from the servo processor 13 and the predetermined basic pressure p₀ are added together by means of the summation unit 11 to obtain the target treatment pressure p_{act} for processing by the pressure regulating device 10.

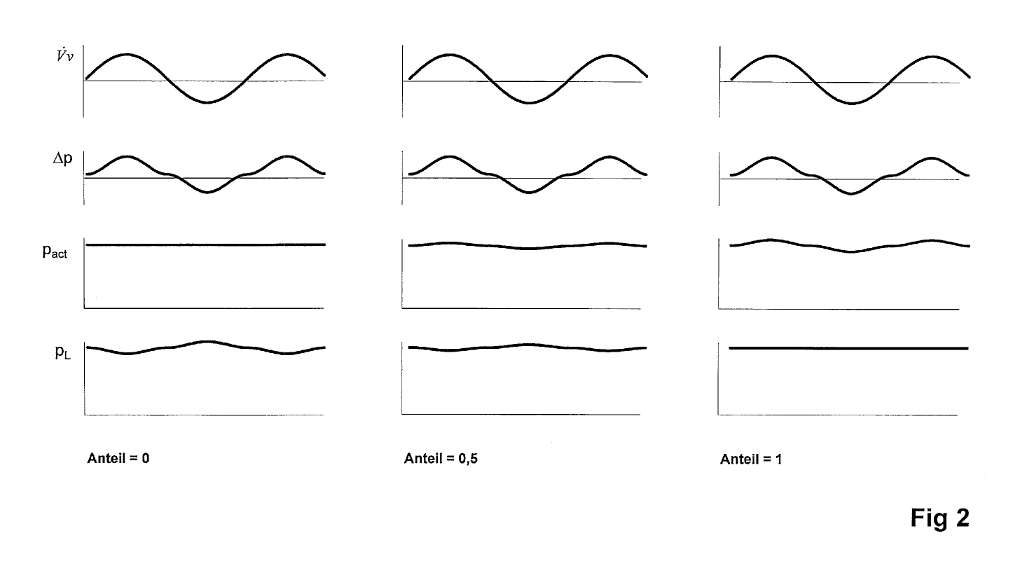

FIG. 2 shows the mode of operation of the respiratory device 1 of the invention in terms of signals. To facilitate understanding of the processes, these are schematically illustrated, and sinusoidal excitations are considered.

Once the respiratory pump P of the patient 2 is describing inspiration and expiration cycles, the capacity (compliance) C of the lungs is being loaded and unloaded via a respiratory airflow \dot{V}ν. In this, the respiratory airflow \dot{V}ν flows through the airway resistance R_{AW}, whereby a portion of the treatment pressure p_{act} is consumed. Between the pressure consumption Δp and the airflow, there is an approximately quadratic rather than a linear connection. As the figure shows, the chronological pattern of the pressure consumption Δp is therefore no longer sinusoidal in shape.

With the respiratory device of the invention, the treatment pressure p_{act} is continuously being adjusted by a portion of the pressure loss Δp.

If this portion is zero (represented on the left of the figure), the treatment pressure p_{act} is unchanged over time. In this case, the periodically fluctuating pattern of the pressure loss Δp that is elicited by inspiration and expiration manifests itself entirely in fluctuations in the intrapulmonary pressure p_{L}. To a person using the respiratory device, this means that he must overcome the respiratory obstructions caused by his respiratory resistance R_{AW} using only his respiratory musculature.

With an increasing portion (represented at the center (portion=0.5) and right (portion=1) of the figure), the periodic pattern of the pressure loss Δp elicited by inspiration and expiration increasingly causes fluctuations in the treatment pressure p_{act}. Consequently, fluctuations in the intrapulmonary pressure p_{L} decrease to the same degree. For the user, this means that his respiratory efforts decrease. The respiratory effort required to overcome the airway resistance R_{AW} is taken over to an increasing degree by the pressure generator. Even a user with an elevated airway resistance R_{AW} will then require no great effort from the respiratory musculature for normal inspiration or expiration processes. He can realize equal ventilation intensity with much lower intrapulmonary suction or pressure.

To determine the level of airway resistance R_{AW}, the process of oscillatory respiratory resistance measurement can be used, for example.

Another possibility consists in determining the airway resistance using external measuring processes and inputting the measured or otherwise determined values.

With the respiratory device of the invention, obstructive sleep apneas can be treated by generating an artificial atmosphere with the help of the pressure generator 3, which is supplied to the patient 2 via the respiratory tube 6 and the breathing mask 7. In this treatment, the respiratory device 1 adjusts the pressure of the artificial atmosphere such that the predetermined treatment pressure p_{act} of the artificial atmosphere is changed by a proportional amount of the consumed pressure Δp at an airway resistance R_{AW} in the airway of the patient. Preferably, the proportional amount by which the treatment pressure p_{act} of the artificial atmosphere is adjusted, is separately predetermined for an inspiration or an expiration.

Although the invention herein has been described with reference to particular embodiments, it is to be understood that these embodiments are merely illustrative of the principals and applications of the present invention. Accordingly, while the invention has been described with reference to the structures and processes disclosed, it is not confined to the details set forth, but is intended to cover such modifications or changes as may fall within the scope of the following claims.

This application incorporates by reference German Patent Application No. 10 2006 032 620.2, filed in the German Patent Office on Jul. 13, 2006.

### LIST OF REFERENCE SYMBOLS

1 Respiratory device
2 Patient
3 Pressure generator
4 Oscillation pump
5 Summation unit
6 Respiratory tube
7 Breathing mask
8 Flow processor
9 Pressure processor
10 Pressure regulating device
11 Summation unit
12 Reversing switch
13 Servo processor
A Outlet
B Outlet
C Capacity of the lungs (compliance)
Int Intensity
P Respiratory pump
P₀ Basic pressure
p_{act} Treatment pressure
p_{L} Intrapulmonary pressure
R_{AW} Airway resistance
R_{AWX} Externally determined airway resistance
Ri Flow resistance
R_{L} Flow resistance
\dot{V}_{D} Device airflow
\dot{V}_{L} Leakage airflow
\dot{V}ν Respiratory airflow
Δp Pressure loss

1. A respiratory device for treating obstructive sleep apnea comprising a pressure generator for generating an artificial atmosphere at a treatment pressure, a pressure regulating device for controlling said pressure generator, a respiratory tube with a breathing mask for supplying the artificial atmosphere generated by the pressure generator to a patient, and a control mechanism for continuously determining a pressure loss that results from an airway resistance of the patient and for the essentially proportional adjustment of the treatment pressure pattern to the pattern of the determined pressure loss.

2. A respiratory device according to claim 1, further comprising a device for continuously determining the current respiratory airflow, and wherein the control mechanism is equipped to continuously determine the airway resistance is from the values for respiratory airflow and airway resistance.

3. A respiratory device according to claim 1, wherein the control mechanism is a servo processor.

4. A respiratory device according to claim 1, further comprising a device for the permanent or intermittent, automatic determination of the airway resistance.

5. A respiratory device according to claim 1, further comprising a device that operates according to the principle of an auto-CPAP, to automatically determine an optimal basic pressure.

6. A method for controlling a respiratory device controlling a pressure generator for generating the treatment pressure by a control mechanism such that the treatment pressure of the artificial atmosphere is adjusted based upon a predetermined basic pressure, changing said treatment pressure by a portion of the actually determined pressure consumed at an airway resistance in the airway of the patient.

7. A method according to claim 6, further comprising an intensity signal in advance to the control mechanism, which signal determines whether the entire pressure loss that is consumed over the airway resistance of the patient or only a portion of it should be compensated for by the respiratory device.

8. A method according to claim 7, further comprising adding an initial value from the control mechanism to a value for the predetermined basic pressure by summation unit, and supplying the obtained summation signal to the pressure regulating device as a command variable for a treatment pressure to be regulated by the pressure generator.
